(19) United States
(12) Patent Application Publication
(10) Pub. No.: US 2014/0294967 A1
(43) Pub. Date: Oct. 2, 2014

(54) STABLE NANOCOMPOSITION COMPRISING PACLITAXEL, PROCESS FOR THE PREPARATION THEREOF, ITS USE AND PHARMACEUTICAL COMPOSITIONS CONTAINING IT

(71) Applicants: János BORBÉLY, Debrecen (HU); Zoltán KÖRHEGYI, Kondó (HU); Krisztina KEREKES, Göd (HU); Magdolna BODNÁR, Debrecen (HU)

(72) Inventors: János BORBÉLY, Debrecen (HU); Zoltán KÖRHEGYI, Kondó (HU); Krisztina KEREKES, Göd (HU); Magdolna BODNÁR, Debrecen (HU)

(73) Assignee: BBS Nanotechnology Llc., Debrecen (HU)

(21) Appl. No.: 14/228,709

(22) Filed: Mar. 28, 2014

Related U.S. Application Data
(60) Provisional application No. 61/805,950, filed on Mar. 28, 2013.

(51) Int. Cl. A61K 47/36; A61K 47/18; A61K 47/22; A61K 31/337

(52) U.S. Cl. CPC A61K 47/36; A61K 31/337; A61K 47/183; A61K 47/22
USPC 424/489; 514/449

(57) ABSTRACT

A nanoparticulate composition is disclosed for the targeted therapeutic treatment of tumours. The stable self assembled nanocomposition according to the invention comprises (i) a carrier and targeting system comprising an optionally modified polyanion, and optionally a polycation, which may also be modified; at least one targeting agent which is linked to either the polycation/modified polycation or the polyanion/modified polyanion, or both or to the surface of the nanoparticle; (ii) paclitaxel as active compound; and optionally (iii) at least one complexing agent, a metal ion and a stabilizer/formulating agent or a PEGylating agent. The invention furthermore relates to a process for the preparation of the above-mentioned composition, the therapeutic uses thereof, and pharmaceutical compositions containing the nanocomposition according to the invention.

This application claims priority to U.S. provisional application Ser. No. 61/805,950, filed Mar. 28, 2013, the entire disclosure of which is hereby incorporated by reference herein.

### FIELD OF THE INVENTION

The present invention relates to a nanoparticulate composition for the targeted therapeutic treatment of tumours. The stable self assembled nanocomposition according to the invention comprises (i) a carrier and targeting system comprising an optionally modified polyanion, and optionally a polycation, which may also be modified; at least one targeting agent which is linked to either the polycation/modified polycation or the polyanion/modified polyanion,or both or to the surface of the nanoparticle; (ii) paclitaxel as active compound; and optionally (iii) at least one complexing agent, a metal ion and a stabilizer/formulating agent or a PEGylating agent. The present invention furthermore relates to a process for the preparation of the above-mentioned composition, the therapeutic uses thereof, and pharmaceutical compositions containing the nanocomposition according to the invention.

### BACKGROUND OF THE INVENTION

Paclitaxel, (2α,4α,5β,7β,10β,13α)-4,10-bis(acetyloxy)-13-{[(2R,3S)-3-(benzoylamino)-2-hydroxy-3-phenylpropanoyl]oxy}-1,7-dihydroxy-9-oxo-5,20-epoxytax-11-en-2-yl benzoate, the compound according to Formula I, is a drug used in cancer chemotherapy.

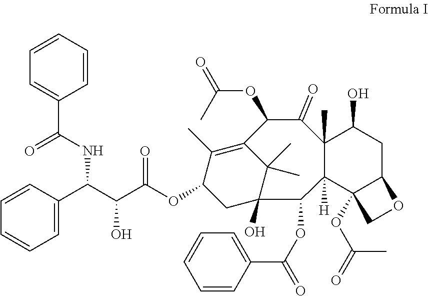 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Paclitaxel (PACL) is used for for ovarian, breast and lung cancers and Kaposi's sarcoma. Paclitaxel is one of several cytoskeletal drugs that target tubulin. Paclitaxel-treated cells have defects in mitotic spindle assembly, chromosome segregation, and cell division. Unlike other tubulin-targeting drugs such as colchicine that inhibit microtubule assembly, paclitaxel stabilizes the microtubule polymer and protects it from disassembly. Chromosomes are thus unable to achieve a metaphase spindle configuration. This blocks progression of mitosis, and prolonged activation of the mitotic checkpoint triggers apoptosis or reversion to the G-phase of the cell cycle without cell division.

Common side effects include nausea and vomiting, loss of appetite, change in taste, thinned or brittle hair, pain in the joints of the arms or legs lasting two to three days, changes in the color of the nails, and tingling in the hands or toes. More serious side effects such as unusual bruising or bleeding, pain/redness/swelling at the injection site, change in normal bowel habits for more than two days, fever, chills, cough, sore throat, difficulty swallowing, dizziness, shortness of breath, severe exhaustion, skin rash, facial flushing, female infertility by ovarian damage and chest pain can also occur. However a number of these side effects are associated with the excipient used, Cremophor EL, a polyoxyethylated castor oil.

### DESCRIPTION OF THE STATE OF THE ART

The problem to be solved in a great number of the chemotherapeutic treatments is the non-specific effect, which means that the chemotherapeutics used is also incorporated in the sane cells and tissues, causing their death. As it can be seen above, the adverse effects of paclitaxel cause a limiting factor for the dosing regimen. There is an unmet need to find a composition comprising a carrier and targeting system, which delivers the active compound specifically to the tumour cells, thereby reducing the dose needed, and accordingly, the adverse effects on the intact tissues.

A number of attempts have been made to find a composition which satisfies the above need. U.S. Pat. No. 7,976,825 discloses a macromolecular contrast agent for magnetic resonance imaging. Biomolecules and their modified derivatives form stable complexes with paramagnetic ions thus increasing the molecular relaxivity of carriers. The synthesis of biomolecular based nanodevices for targeted delivery of MRI contrast agents is also described. Nanoparticles have been constructed by self-assembling of chitosan as polycation and poly-gamma glutamic acids as polyanion. Nanoparticles are capable of Gd-ion uptake forming a particle with suitable molecular relaxivity. There is no active agent and therapeutic use disclosed in U.S. Pat. No. 7,976,825.

U.S. Pat. No. 8,007,768 relates to a pharmaceutical composition of the nanoparticles composed of chitosan, a negatively charged substrate, a transition metal ion, and at least one bioactive agent for drug delivery. The nanoparticles are characterized with a positive surface charge configured for promoting enhanced permeability for bioactive agent delivery. The pharmaceutical composition consists of a shell portion that is dominated by positively charged chitosan and a core portion, wherein the core portion consists of the positively charged chitosan, a transition metal ion, one negatively charged substrate, at least one bioactive agent loaded within the nanoparticles, and optionally a zero-charge compound. The composition may contain at least one bioactive agent selected from the group of exendin-4, GLP-1, GLP-1 analog, insulin or insulin analog. Paclitaxel is not mentioned among the possible active agents.

WO2007019678 relates to an implantable device comprising a biocompatible and biodegradable matrix impregnated with a bioactive complex suitable for selectively targeting the lymphatic system, wherein the bioactive complex comprises one or more particle forming materials and among other bioactive agents e.g. paclitaxel. The implantable device according to the document comprises a biocompatible and biodegradable matrix impregnated with a bioactive complex suitable for selectively targeting the lymphatic system, wherein the bioactive complex comprises one or more particle forming materials and one or more bioactive agents. The particles are microparticles or nanoparticles or their combination of microparticles and nanoparticles and the particle size is from about 0.3 μm to about 11.2 μm. Unlike our invention, there is no targeting agent in the above-mentioned composition, and the specific effect is attempted to be achieved by implantation.

US2006073210 relates to a method of enhancing intestinal or blood brain paracellular transport configured for delivering at least one bioactive agent in a patient comprising administering nanoparticles composed of [gamma]-PGA and chitosan. The administration of the nanoparticles takes place orally. The chitosan is a low molecular weight chitosan (50 kDa) and dominates on a surface of said nanoparticles. The surface of said nanoparticles is characterized by a positive surface charge. The nanoparticles have a mean particle size between about 50 and 400 nanometers and are formed via a simple and mild ionic-gelation method. The nanoparticles are loaded with a therapeutically effective amount of at least one bioactive agent. In the above-mentioned prior art document paclitaxel is not mentioned as possible therapeutically active agent. Furthermore, though the composition may enhance the penetration of the blood brain carrier, targeting of the therapeutics has not been solved by the invention.

WO06042146 relates to conjugates comprising a nanocarrier, a therapeutic agent or imaging agent and a targeting agent. Among others, the use of polyglutamic acid, chitosan or combinations thereof as nanocarriers, for the delivery of gadolinium as a contrast agent, or for delivering paclitaxel or paclitaxel as chemotherapeutic agents is described. According to the document, the use of gadolinium serves solely diagnostic purposes, complexing agent is not used to increase the stability of the nanoparticles, and so the use of metal ions to increase the rate of nanoparticles' penetration into targeted cells is not disclosed.

The state of the art failed to solve the above-mentioned problem that is the reduction of the adverse effects of paclitaxel through the decrease of the incorporated active agent by its targeted delivery. There is an unsatisfied need to provide for a stable composition for the targeted therapeutic treatment of tumours using paclitaxel. We performed systematic research in the field and, as a result of our surprising findings, completed our invention.

### BRIEF DESCRIPTION OF THE DRAWINGS

FIG. 1: size distribution by volume.

FIG. 2a-c: MTT results.

FIG. 3a-c: HeLa, A2780 and KB cells measured by Real Time Analyser (Roche).

FIG. 4a-c: MTT results.

FIG. 5: Preparation of targeting, paclitaxel loaded, self-assembled nanoparticles.

### DETAILED DESCRIPTION OF THE INVENTION

We have surprisingly found that a stable, self assembling nanocomposition may be prepared by using a polycation together with a polyanion when preparing the carrier of the pharmaceutically active agent. The nanocarrier system according to the present invention consists of at least four components: a polycation, a polyanion, an active agent, which is paclitaxel, and a targeting molecule, which may be linked to the polycation, the polyanion or both. The composition may additionally contain a complexing agent bound covalently to the polycation, a metal ion, and a stabilizer/formulating agent, or a PEGylating agent, though these are not necessarily included the composition. The formation of the nanoparticles takes place by the self assembling of the polyelectrolites.

Accordingly, in its first aspect the invention relates to a stable self assembled composition comprising

(i) a carrier and targeting system comprising an optionally modified polyanion, and optionally a polycation, which may also be modified; at least one targeting agent which is linked to either the polycation/modified polycation or the polyanion/modified polyanion, or both or to the surface of the nanoparticle;

(ii) paclitaxel as active compound; and optionally

(iii) at least one complexing agent, metal ion and formulating agent.

In a preferred embodiment, the biopolymers are water-soluble, biocompatible, biodegradable polyelectrolyte biopolymers.

One of the polyelectrolyte biopolymers is a polycation, positively charged polymers, which is preferably chitosan (CH) or any of its derivatives. E.g. in the composition according to the invention the polycation may be chitosan, the modified polycation may be selected from the derivatives of chitosan, especially chitosan-EDTA, chitosan-DOTA, chitosan-DTPA, chitosan-FA, chitosan-LHRH, chitosan-RGD, however, they are not limited thereto.

The other type of the polyelectrolyte biopolymers is a polyanion, a negatively charged biopolymer. Preferably the polyanion is selected from the group of poly-gamma-glutamic acid (PGA), polyacrylic acid (PAA), hyaluronic acid (HA), alginic acid (ALG), and the modified derivatives thereof.

The derivatives of biopolymers can be their cross-linked nanosystems, biopolymer-complexone conjugates, targeting agent—biopolymer product or other grafted derivatives resulted in modifications of biopolymers with other molecules, e.g. polyethylene glycol (PEG) oligomers.

Preferably the complexing agent is selected from the group of diethylenetriaminepentaacetic acid (DTPA), 1,4,7,10-tetracyclododecane-N,—N′,N″,N′″-tetraacetic acid (DOTA), ethylene-diaminetetraacetic acid (EDTA), 1,4,7,10-tetraazacyclododecane-N,N′,N″-triacetic acid (DO3A), 1,2-diaminocyclohexane-N,N,N′,N′-tetraacetic acid (CHTA), ethylene glycol-bis(beta-aminoethylether)N,N,N′,N′,-tetraacetic acid (EGTA), 1,4,8,11-tetraazacyclotradecane-N,N′,N″,N′″-tetraacetic acid (TETA), and 1,4,7-triazacyclononane-N,N′,N″-triacetic acid (NOTA), but is not limited to these materials.

Preferably the targeting agent is selected from the group of small molecules, preferably folic acid (FA), octreotide (OCT) peptides, preferably luteinsing hormone releasing hormone (LHRH), arginin-glycin-aspartate amino acid sequence (RGD), a monoclonal antibody, preferably Transtuzumab.

In a preferred embodiment, the drug molecules are ionically or covalently attached to the bioanion or the biocation or its derivatives via their functional groups. In case of covalent conjugation, water-soluble carbodiimide, as coupling agent is used to make stable amide bonds between the drug molecules and the biopolymers via their carboxyl and amino functional groups in aqueous media.

The metal ion is selected from the group of calcium, magnesium, copper, gallium, gadolinium or manganese ion; and the formulating agent is selected from the group of glucose, physiological salt solution, PBS or any combination thereof.

As used in the present invention the abbreviations below have the following meanings:

PGA means poly-gamma-glutamine acid

PAA means polyacrylic acid

HA means hyaluronic acid

ALG means alginic acid

CH means chitosanFA means folic acid

OCT means octreotide

LHRH means luteinsing hormone releasing hormone

RGD means arginin-glycin-aspartate amino acid sequence

PACL means paclitaxel

DTPA means diethylene-triamine-pentaacetic acid

DOTA means 1,4,7,10-tetracyclododecane-N,—N′,N″,N′″-tetraacetic acid

EDTA means ethylene-diaminetetraacetic acid

DO3A means 1,4,7,10-tetraazacyclododecane-N,N′,N″-triacetic acid

CHTA means 1,2-diaminocyclohexane-N,N,N′,N′-tetraacetic acid

EGTA means ethylene glycol-bis(beta-aminoethylether)N,N,N′,N′,-tetraacetic acid

TETA means 1,4,8,11-tetraazacyclotradecane-N,N′,N″,N′″-tetraacetic acid

NOTA means 1,4,7-triazacyclononane-N,N′,N″-triacetic acid

PGA-FA means poly-gamma-glutamic acid-bound folic acid

PGA-PACL means poly-gamma-glutamic acid-bound paclitaxel

PGA-FA-PACL means folic acid-PGA-bound paclitaxel

PGA-LHRH means poly-gamma-glutamic acid-bound luteinsing hormone releasing hormone

PGA-RGD means poly-gamma-glutamic acid-bound arginin-glycin-aspartate amino acid sequence

PAA-FA means polyacrylic acid-bound folic acid

PAA-LHRH means polyacrylic acid-bound luteinsing hormone releasing hormone

PAA-RGD means polyacrylic acid-bound arginin-glycin-aspartate amino acid sequence

HA-FA means hyaluronic acid-bound folic acid

HA-RGD hyaluronic acid-bound arginin-glycin-aspartate amino acid sequence

HA-LHRH hyaluronic acid-bound luteinsing hormone releasing hormone

ALG-FA means alginic acid-bound folic acid

ALG-LHRH means alginic acid-bound luteinsing hormone releasing hormone

ALG-RGD means alginic acid-bound arginin-glycin-aspartate amino acid sequence

CH-EDTA means chitosan-bound ethylene-diaminetetraacetic acid

CH-DOTA means chitosan.-bound 1,4,7,10-tetracyclododecane-N,—N′,N″,N′″-tetraacetic acid CH-DTPA means chitosan-bound diethylene-triamine-pentaacetic acid

CH-FA means chitosan-bound folic acid

CH-LHRH means chitosan-bound luteinsing hormone releasing hormone

CH-RGD means chitosan-bound arginin-glycin-aspartate amino acid sequence

EDC*HCL means (1-ethyl-3-(3-dimethylaminopropyl)-carbodiimide methiodide)

DMSO means dimethyl-sulphoxide

NaOH means sodium-hydroxide

PA means polyanion

PC means polycation

NP means nanoparticle

HOBt means 1-hydroxybenzotriazole hydrate

TEA means tryethylamine

PEG means polyethylene glycol

FA-PEG-NH₂ means folic acid polyethylene glycol amine

FA-PEG means pegylated folic acid

PGA-PEG-FA means poly-gamma-glutamic acid bound pegylated folic acid

PGA-PEG-FA-PACL means paclitaxel loaded PGA-PEG-FA

PGA-PEG-FA-OCT means octreotide loaded PGA-PEG-FA

NP-PACL means PACL loaded NP, targeting agent: FA

NP-PACL-OCT means PACL loaded NP, targeting agent: FA and OCT

A preferred self-assembled composition according to the present invention is characterized by any one or more of the following features:

(i) the average size of the nanoparticles in swollen state is in the range between 30 to 500 nm, preferably 60 to 200 nm, more preferably about 80 to 120 nm;

(ii) the proportion of the polycation to the polyanion is about 1:20 to 20:1 based on the weight of the agents;

(iii) the polyanion has a pH of 7.5 to 10; a molecular weight of 10 000 Da to 1.5 MDa and a concentration of 0.01 to 2 mg/ml;

(iv) the polycation has a pH of 3.5 to 6; a molecular weight of 60 to 320 kDa and a concentration of 0.01 to 2 mg/ml.

In its second aspect the present invention relates to a process for the preparation of the above mentioned composition according to the invention, characterized in that it comprises the steps of

(i) a targeting agent is bound covalently to the polycation and/or the polyanion;

(ii) the active agent is bound covalently or by an ionic bond to the polycation and/or the polyanion;

(iii) the polycation and the poly anion are contacted with each other, preferably in a ratio of 1:20 to 20:1 based on the weight of the agents, thus are reacted with each other to self-assemble;

(iv) optionally the other components are added to the reaction mixture.

In a preferred embodiment the polyanion used in the process according to the invention has a pH of 7.5 to 10; a molecular weight of 10 000 Da to 1.5 MDa and a concentration of 0.01 to 2 mg/ml; and the polycation used has a pH of 3.5 to 6; a molecular weight of 60 to 320 kDa and a concentration of 0.01 to 2 mg/ml.

Prior to the reaction of the polyelectrolites any one of them or all of them is/are bound to a targeting agent by a covalent bond, thus the nanoparticles will cumulate in the tumourous cells. Furthermore, an active agent according to the present invention is bound to the polycation and/or the polyanion, either by covalent or by ionic bond. It is critical to form such a bond between the active compound and the polycation and/or the polyanion, which is likely to be split by the time of being incorporated in the target cell, and the active compound is released.

On reaction of the polycation and the polyanion a self-assembly takes place, contracting the molecule and resulting in a stable nanosystem. The thus formed nanoparticles possess negative surface charge and a narrow range of size distribution, which ensure the uniform physical and chemical characteristics. The resulting composition is a hydrophilic nanosystem, and forms stable colloid systems in water.

The nanosystem can be designed to achieve compositions with exactly expected features. The type of the self-assembling biopolymers, the order of admixing of the polycation and the polyanion (or their modified derivatives), the molecular weight, the mass ratio, the concentration and the pH of the the polycation and the polyanion (or their modified derivatives) will result in different features (size, surface charge, active agent content, targeting agent content, etc.) of the system. The selection of the above elements may be done by the skilled person, knowing the object without undue experimentation.

Furthermore, the present invention relates to a stable self-assembled composition comprising

(i) a carrier and targeting system comprising an optionally modified polycation, and an optionally modified polyanion; at least one targeting agent which is linked to either the polycation/modified polycation or the polyanion/modified polyanion, or both;

(ii) paclitaxel as active compound; and optionally

(iii) at least one complexing agent, metal ion and formulating agent,

which is obtainable by the above-mentioned process according to the invention.

In its third aspect the invention relates to a pharmaceutical composition comprising the composition according to the invention together with pharmaceutically acceptable auxiliary materials, preferably selected from group of glucose, physiological salt solution, and PBS.

Furthermore, the present invention relates to the use of the composition according to the invention or the pharmaceutical composition according the invention for the preparation of a medicament; and the use of the composition or the pharmaceutical composition according to the invention for the treatment of tumours. Finally the invention relates to a method for the treatment of a subject in need for the treatment of tumours, especially human cervical adenocarcinoma, human ovary carcinoma, human breast carcinoma, human lung adenocarcinoma, human cervical carcinoma, human skin melanoma, human colon adenocarcinoma and human prostate carcinoma by administering to the subject an effective amount of the composition or the pharmaceutical composition according to the present invention.

### EXAMPLES

Preparation of the Formulation According to the Invention

Tests of the Effectiveness of the Compositions According to the Invention

The internalization and accumulation of the nanosystem according to the present invention were proved on different cell lines in vitro; the cytotoxicity of the nanosystem was tested by investigating the viability of the cells using the MTT method, on among others human cervical adenocarcinoma (HeLa), human ovary carcinoma (A2780, SK-OV-3), human prostate carcinoma (PC-3, LNCaP), human breast carcinoma (MCF-7, MDA-MB231), human lung adenocarcinoma (A549, H1975), human cervical carcinoma (KB), human skin melanoma (HT168-M1/9), human colon adenocarcinoma (HT29), human melanoma (WM983A) and human metastatic melanoma (WM983B) cell line

The drug-loaded nanosystems are stable at pH 7.4, it may be injected intravenously. Based on the blood circulation, the nanoparticles could be transported to the area of interest.

The osmolarity of nanosystem was adjusted to the value of human serum. In a preferred embodiment, the osmolarity was set using formulating agents, selected from the group of glucose, physiological salt solution.

The effects of glucose, physiological saline solution, infusion base solutions and different buffers on the size, size distribution and stability of the nanoparticles were investigated.

The xCELLigence RTCA HT Instrument from Roche Applied Science uses gold electrodes at the bottom surface of microplate wells as sensors to which an alternating current is applied. Cells that are grown as adherent monolayers on top of such electrodes influence the alternating current at the electrodes by changing the electrical resistance (impedance). The degree of this change is primarily determined by the number of cells, strength of the cell-cell interactions, interactions of the cells with the microelectrodes and by the overall morphology of the cells. The RTCA Software calculates the Cell Index (CI) as the relative change in measured impedance to represent cell status. The normalized cell index (NCI—plotted on y axis) is the relative cell impedance presented in the percentage of the value at the base-time. NCI shows rate of the surface covered by cells. NCI increases by rise of cell-number or cell-size. For example NCI value in a culture treated with a proliferation inhibitory drug first can increase (because the cell-size grows) and after decreases (because the cell-number reduces).

The MTT test is a colorimetric assay that measures the reduction of yellow 3-(4,5-dimethythiazol-2-yl)-2,5-diphenyl tetrazolium bromide (MTT) by mitochondrial succinate dehydrogenase. The MTT enters the cells and passes into the mitochondria where it is reduced to an insoluble, coloured (dark purple) formazan product. The cells are then solubilised with an organic solvent (dimethyl sulfoxide) and the released, solubilised formazan reagent is measured spectrophotometrically. Since reduction of MTT can only occur in metabolically active cells the level of activity is a measure of the viability of the cells. This method can therefore be used to measure cytotoxicity, proliferation or activation.

Cell Lines:

| Cell line | Type of carcinomacell |
|---|---|
| HeLa | Human cervicaladenocarcinomacell line |
| A2780 | Human ovarycarcinoma cell line |
| SK-OV-3 | Human ovary adenocarcinoma cell line |
| A549 | Human lung adenocarcinoma cell line |
| H1975 | Human lung adenocarcinoma cell line |
| JIMT-1 | Human breastcancer cell line |
| MCF-7 | Human breastcarcinoma cell line |
| PC-3 | Human prostatecarcinoma cell line |
| LNCaP | Human prostatecarcinoma cell line |
| KB | Human cervicalcarcinoma cell line |
| MDA-MB-231 | Human breastcarcinoma cell line |
| HT29 | Human colon adenocarcinoma cell line |
| WM983A | Human melanoma cell line |
| WM983B | Human metastaticmelanoma cell line |

### EXAMPLES

### Example 1

### Preparation of Folated Poly-Gamma-Glutamic Acid (γ-PGA)

Folic acid was conjugated via the amino groups to γ-PGA using carbodiimide technique. γ-PGA (m=50 mg) was dissolved in water (V=50 ml) to produce aqueous solution. After the addition of 1-[3-(dimethylamino)propyl]-3-ethylcarbodiimide methiodide (EDC*HCl) (m=22 mg) to the γ-PGA aqueous solution, the reaction mixture was stirred at 4° C. for 30 min After that, folic acid (m=32 mg in dimethyl sulfoxide, V=10 ml) was added dropwise to the reaction mixture and stirred at room temperature for 24 h. The folated poly-γ-glutamic acid (PGA-FA) was purified with membrane filtration.

### Example 2

### PEG-Folic Acid Association with PGA

Poly-gamma-glutamic acid (m=300 mg) was solubilized in water (V=300 ml), then HOBt (m=94 mg) was added to the PGA solution. The solution was stirred at 4° C. for 15 minutes, then 1-(3-dimethylaminopropyl)-3-ethylcarbodiimide hydrochloride (EDC*HCl) (m=445 mg in 15 ml water) was added to the solution. The mixture was stirred for 10 minutes while cooling on ice, then folic acid-PEG-amine (NH₂-PEG-NH-FA) (m=100 mg in 10 ml water) and TEA (m=235 mg) was added to the reaction mixture and stirred at room temperature in the dark for 24 hours. The PGA-FA-PEG was purified with membrane filtration.

The preparation of the PGA-PEG-FA is illustrated by the reaction scheme below.

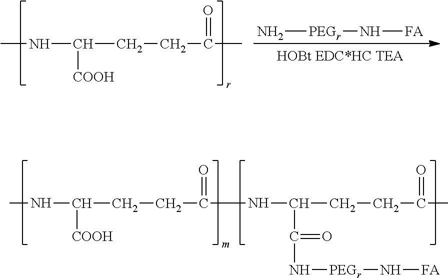

### Example 3

### Preparation of Folated Chitosan

A solution of 1-(3-dimethylaminopropyl)-3-ethylcarbodiimide hydrochloride (CDI) and FA in anhydrous DMSO was prepared and stirred at room temperature until FA was well dissolved (1 h).Chitosan was dissolved in 0.1 M hydrochloric acid, to produce a solution with a concentration of 1 mg/ml, and then adjusted to pH 5.5 with 0.10 M sodium hydroxide solution. After the dropwise addition of EDC*HCl (m=5.1 mg in 1 ml distilled water) to the chitosan solution (V=20 ml), the reaction mixture was stirred for 10 min. Then folic acid (m=8.5 mg in dimethyl sulfoxide, V=1 ml) was added to the reaction mixture. The resulting mixture was stirred at room temperature in the dark for 24 h. It was brought to pH 9.0 by drop wise addition of diluted aqueous NaOH and was washed three times with aqueous NaOH, and once with distilled water. The polymer was isolated by lyophilization.

### Example 4

### Preparation of Chitosan-DTPA Conjugate

Chitosan (m=15 mg) was solubilized in water (V=15 ml); its dissolution was facilitated by dropwise addition of 0.1 M HCl solution. After the dissolution, the pH of chitosan solution was adjusted to 5.0. After the dropwise addition of DTPA aqueous solution (m=11 mg, V=2 ml, pH=3.2), the reaction mixture was stirred at room temperature for 30 min, and at 4° C. for 15 min. after that, EDC*HCl (m=8 mg, V=2 ml distilled water) was added dropwise to the reaction mixture and stirred at 4° C. for 4 h, then at room temperature for 20 h. The chitosan-DTPA conjugate (CH-DTPA) was purified by dialysis.

### Example 5

### Preparation of Paclitaxel Loaded Poly-Gamma-Glutamic Acid—Ionically Bound

Poly-gamma-glutamic acid (m=5.0 mg) was dissolved in water (V=10 ml) and then adjusted to pH 9.0. Paclitaxel (PAC) solution (V=250 μl) with a concentration of c=3 mg/ml was added to the PGA solution and the reaction was stirred for 24 h at room temperature. The paclitaxel-loaded PGA was purified by membrane filtration.

### Example 6

### Preparation of Paclitaxel Loaded Poly-Gamma-Glutamic Acid—Covalently Bound

Poly-gamma-glutamic acid (m=5.0 mg) was dissolved in water (V=10 ml) and then adjusted to pH 6.5. m=1 mg of water soluble carbodiimide was dissolved in water (V=500 μl) and mixed with m=0.44 mg of 1-hydroxybenzotriazole hydrate dissolved in DMSO (V=500 μl) to produce a mixture. PAC solution (c=3 mg/ml, V=250 μl) was added dropwise to the PGA solution, and the reaction was stirred for 30 min at room temperature, and for 10 min at 4 C. After that the mixture of CDI and HOBt was added dropwise to the reaction, and the reaction mixture was stirred at 4° C. for 4 h then at room temperature for 20 h. The PAC-loaded PGA was purified by membrane filtration.

### Example 7

### Preparation of Paclitaxel Loaded Folated-Poly-Gamma-Glutamic Acid (PGA-FA-PACL)—Covalent iv Bound

10 ml 0.5 mg/ml folated-PGA was stirred for 15 minutes paclitaxel was added dropwise to the solution and the reaction was stirred for 30 minutes at room temperature, then for 15 minutes at 4° C. 0.8 mg EDC*HCl was dissolved in 1 ml water and mixed 0.6 mg HOBt dissolved in 1 ml water to produce a mixture. The mixture was added to the reaction and the reaction was stirred at 4° C. for 4 hours then room temperature for 20 hours. The PGA-FA-OCT was purified by membrane filtration.

### Example 8

### Preparation of Octreotide Loaded Folated-Poly-Gamma-Glutamic Acid (PGA-FA-OCT)—Covalently Bound

10 ml 0.5 mg/ml folated-PGA was stirred for 15 minutes octreotide was added dropwise to the solution and the reaction was stirred for 30 minutes at room temperature, then for 15 minutes at 4° C. 1 mg EDC*HCl was dissolved in 1 ml water and mixed 0.6 mg HOBt dissolved in 1 ml water to produce a mixture. The mixture was added to the reaction and the reaction was stirred at 4° C. for 4 hours then room temperature for 20 hours. The OCT-FA-PACL was purified by membrane filtration.

### Example 9

### Preparation of Paclitaxel Loaded Chitosan—Ionically Bound

Chitosan (m=5.0 mg) was dissolved in 0.01 M hydrochloric acid solution, to produce a solution with a concentration of 0.5 mg/ml, and then adjusted to pH 4.0 with c=0.10 M sodium hydroxide solution. Paclitaxel (PAC) solution (V=250 μl) with a concentration of c=3 mg/ml was added to the chitosan solution and the reaction was stirred for 24 h at room temperature. The PAC-loaded chitosan was purified by dialysis.

### Example 10

### Preparation of Targeting, Paclitaxel Loaded, Self-Assembled Poly-Gamma-Glutamic Acid/Chitosan Nanoparticles

Folated PGA solution (c=0.5 mg/mi) and PACL-loaded PGA solution (c=0.5 mg/mi) were mixed at a ratio of 1:1. The pH of mixture was adjusted to 9.5. Chitosan was dissolved in water (c=0.5 mg/mi), and the pH was adjusted to 4.0. Chitosan solution (V=1 ml) was added to the PGA mixture (V=2 ml), and was stirred at room temperature for 15 min

The preparation of targeting, paclitaxel loaded, self-assembled nanoparticles can be seen in FIG. 5. It noted that the nanosystem can be prepared by a number of methods, the scheme is only one example for the preparation of the three phase system.

### Example 11

### Preparation of Targeting, Paclitaxel Loaded, Self-Assembled Poly-Gamma-Glutamic Acid/Chitosan Nanoparticles

PAC-loaded PGA solution was prepared with a polymer concentration of c=0.3 mg/ml. The pH of the solution was adjusted to 9.5. Folated chitosan was dissolved in aqueous medium with a concentration of 0.3 mg/ml, and the pH was adjusted to 4.0. Folated chitosan solution (V=1 ml) was added dropwise to the PAC-loaded PGA solution (V=2 ml) under continuous stirring.

### Example 12

### Preparation of Pegylated NP-s (Pegylation with MeO-PEG-NH₂ 2000 Da)

4.65 mg MeO-PEG-NH₂ was added drop wise to 15 ml paclitaxel loaded NP (c_{polymer}=0.3 mg/ml) and the solution was stirred for 30 minutes at room temperature, then for 15 minutes at 4° C. 1.38 mg EDC*HCl was dissolved in 1 ml distillated water and mixed 0.63 mg HOBt dissolved in 1 ml distillated water to produce a mixture. The mixture and 0.94 mg TEA was added to the reaction. The reaction was stirred at 4° C. for 4 hours then room temperature for 20 hours. The pegylated NP was purified with membrane filtration. The preparation of pegylated NP-s is illustrated by the reaction scheme according to Example 2.

### Example 13

### Characterization of Self-Assembled, Drug-Laded Nanoparticles

The hydrodynamic size and size distribution of particles was measured using a dynamic light scattering (DLS) technique with a Zetasizer Nano ZS (Malvern Instruments Ltd., Grovewood, Worcestershire, UK). This system is equipped with a 4 mW helium/neon laser with a wavelength of 633 nm and measures the particle size with the noninvasive backscattering technology at a detection angle of 173°. Particle size measurements were performed using a particle-sizing cell in the automatic mode. The mean hydrodynamic diameter was calculated from the autocorrelation function of the intensity of light scattered from the particles. Electrokinetic mobility of the nanoparticles was measured in folded capillary cell (Malvern) with a Zetasizer Nano ZS apparatus.

### Example 14

### Cellular Uptake of Self-Assembled, Drug-Laded Nanoparticles

Internalization and selectivity of nanoparticulates was investigated in cultured human cancer cells overexpressing folate receptors by using confocal microscopy and flow cytometry. The samples were imaged on an Olympus FluoView 1000 confocal microscope. Excitation was performed by using the 488 nm line of an Ar ion laser (detection: 500-550 nm) and the 543 nm line of a HeNe laser (detection: 560-610 nm) to image Alexa 488 and Alexa 546 respectively. Images were analyzed using Olympus FV10-ASW 1.5 software package. Flow cytometric analysis (BD FACSArray Bioanalyzer System) was carried out with a single-cell suspension, and only the live cells were gated based on forward and side scatter dot plots.

In Vivo Results

| | Change in | Change in body | Survival |
|---|---|---|---|
| Treatment (total | tumor volume | weight during the | proportion at |
| dose of 6 | (control: | treatment (weight | the end of the |
| injections) | 100%) | at start: 100%) | experiment |
| Control: 5% glucose | 100% ± 35% | 97% ± 7% | 0% |
| PACL (5 mg/kg) | 86% ± 13% | 99% ± 5% | 60% |
| NP-PACL (5 | 62% ± 13% | 96% ± 6% | 100% |
| mg/kg) | | | |
| NP-PACL-OCT | 64% ± 4% | 101% ± 5% | 100% |
| (1.7 mg/kg) | | | |

The table above illustrates the comparative efficacy study in SK-OV-3 s.c. xenograft SCID mouse model of ovary cancer. Tumor was induced in mice by implanting SK-OV-3 human ovary adenocarcinoma cells s.c. in upper region of back of SCID mice and allowing the tumors to develop to appreciable size over 24 days (70 mm3). The comparative efficacy study of six i.v. injection (day 24, 31, 38, 44, 51 and 58) of 5% glucose, paclitaxel (PACL) 5 mg/kg, NP-PACL 5 mg/kg and NP-PACL-OCT 1.7 mg/kg) was evaluated over 72 days. In this table there are: change in tumor volume of mice on 62nd day after tumor inoculation (data represent mean %±SEM of five mice per group), change in body weight of mice on 62nd day after tumor inoculation (Data represent mean±STDEV of five mice per group) and survival proportion at the end of the experiment.

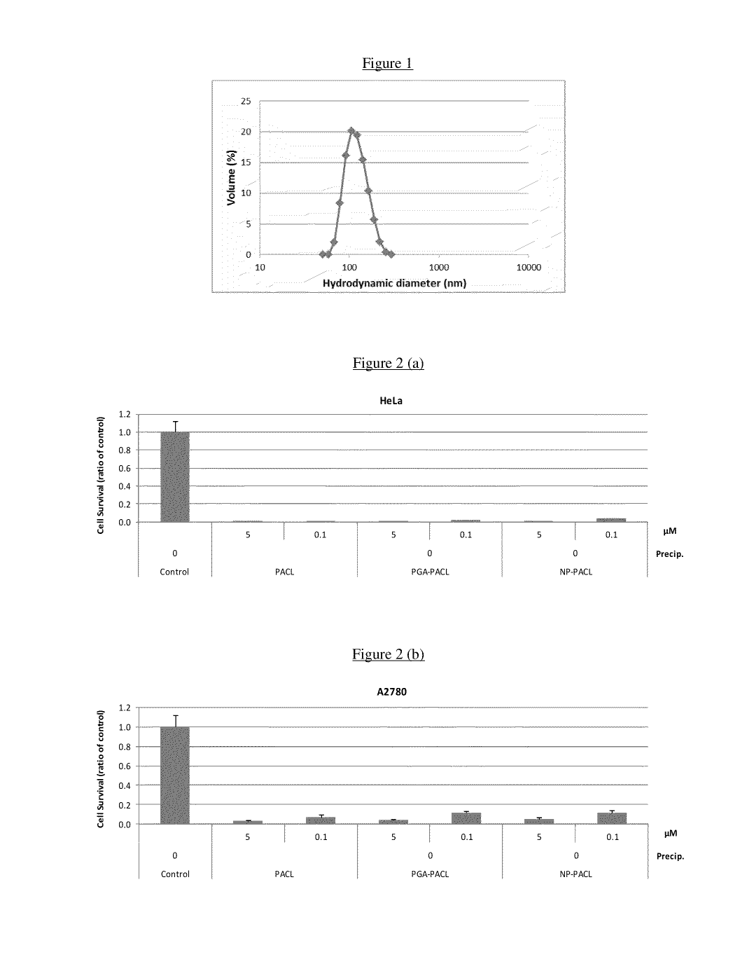

FIG. 1 shows the size distribution of PAC-loaded nanoparticles by volume in which nanocarriers were constructed by self-assembly of biopolymers at a concentration of 0.3 mg/ml, at given ratios, where the CH-PAC solution was added into the PGA-FA solution.

FIG. 2 shows the MTT assay results of PACL drug molecules, PAC-loaded PGA (PGA-PACL) and PAC-loaded nanoparticles (NP-PACL) at different doses using HeLa cell line (a), A2780 cell line (b) and KB cell line (c).

Results of MTT assay confirm that the PAC was successfully conjugated and the PAC-loaded nanoparticles decreased the cell viability of several tumor cells considerably. The viability of tumor cells was investigated in a function of dose of drug-loaded nanoparticles. It was established that folate-targeted PAC-loaded nanoparticles considerably decrease the cell viability depending on the dose of nanoparticles as well as the amount of delivered drug molecules.

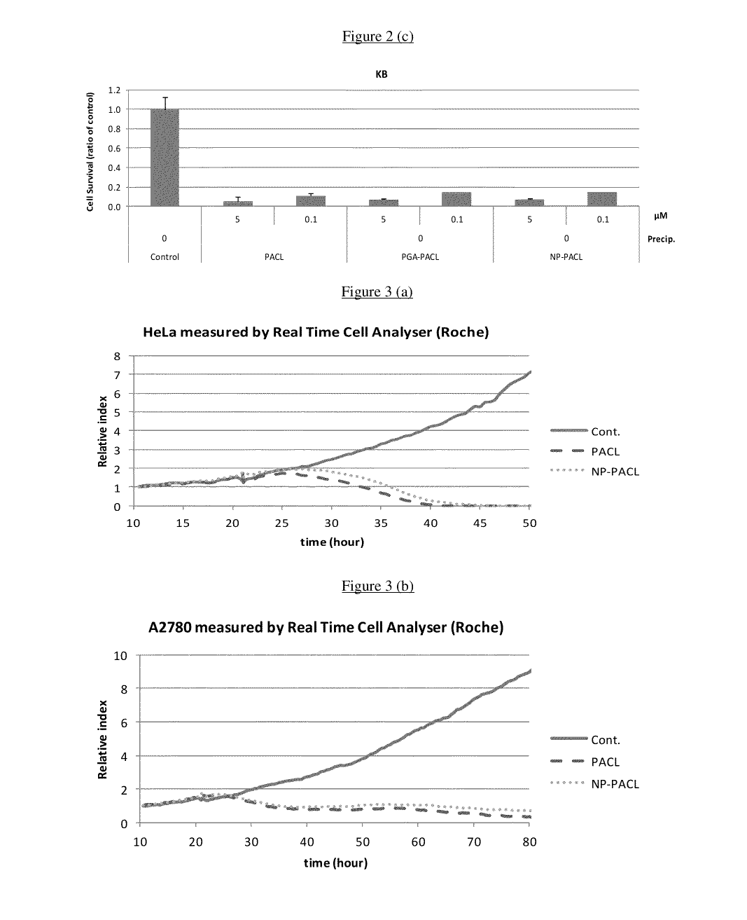

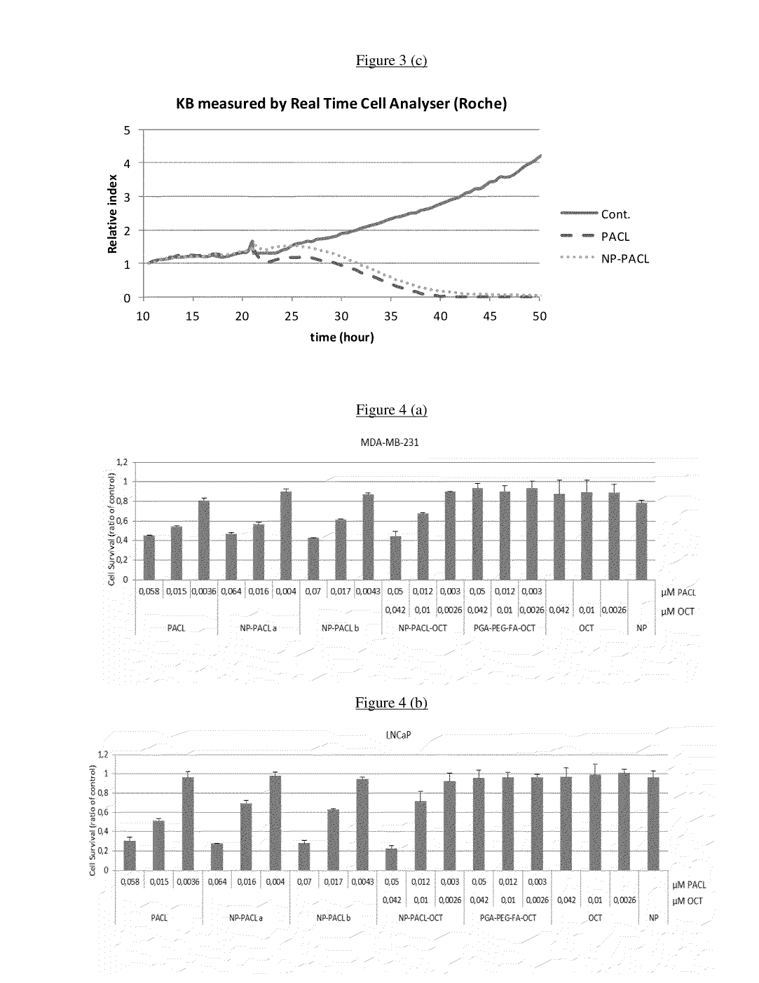

FIG. 3 shows the growth profile of HeLa cells (a), A2780 cells (b), and KB cells (c) after treating with PACL drug molecules (red), PACL-loaded nanoparticles (NP-PACL) (green), and control cells (blue). The injected volume contained the same concentration of paclitaxel.

The results of Roche show that the effect of PACL and PAC-loaded nanoparticles is similar on the studied tumor cell lines; however the nanoparticles due to their targeting ligand deliver the drug molecules into the tumor cells and minimize the side effect of the drug.

Effect of drug was studied for several days. The results support that effect of drug is long-drawn, the living cell index did not increased neither after 3 days.

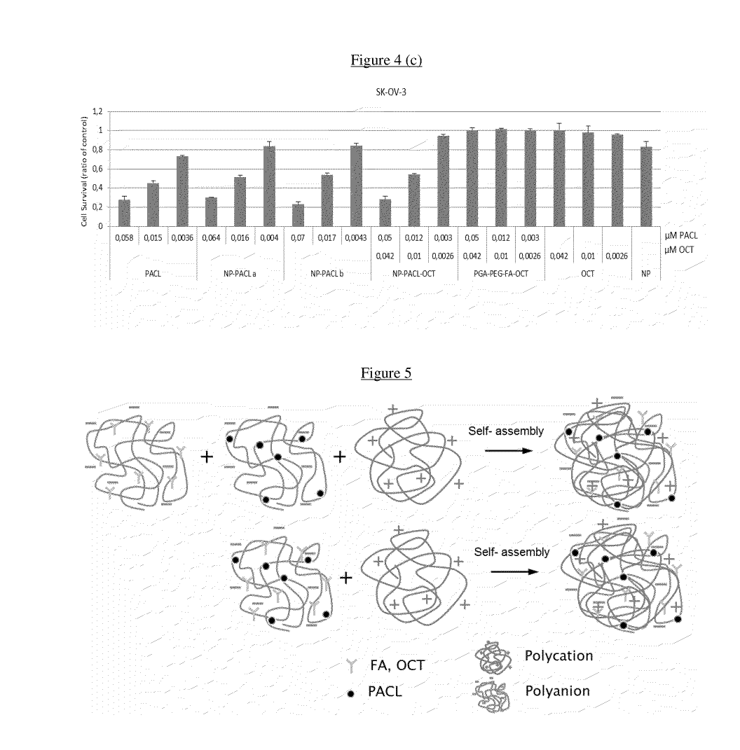

FIG. 4 shows the MTT assay results of PACL drug molecules, PACL-loaded nanoparticles (NP-PACL), NP-PACL-OCT, PGA-PEG-FA-OCT and OCT drug molecule at different doses using MDA-MB-231 cell line (a), LNCaP cell line (b) and SK-OV-3 cell line (c).

FIG. 5. The preparation of targeting, paclitaxel loaded, self-assembled nanoparticles.

1. A stable self-assembled composition comprising
(i) a carrier and targeting system comprising an optionally modified polyanion, and optionally a polycation, which may also be modified; at least one targeting agent which is linked to either the polycation/modified polycation or the polyanion/modified polyanion, both or to the surface of the nanoparticle;
(ii) paclitaxel as active compound and optionally
(iii) at least one complexing agent, metal ion and stabilizer/formulating agent or a PEGylating agent.

2. The composition according to claim 1, wherein the polycation is chitosan, the modified polycation is selected from the group of derivatives of chitosan, especially chitosan-EDTA, chitosan-DOTA, chitosan-DTPA, chitosan-FA, chitosan-LHRH, chitosan-RGD;
the polyanion is selected from the group of poly-gamma-glutamic acid (PGA), polyacrylic acid (PAA), hyaluronic acid (HA), alginic acid (ALG); and the modified derivatives thereof,
the derivatives of biopolymers can be their cross-linked nanosystems, biopolymer-complexone products, or other grafted derivatives resulted in modifications of biopolymers with other molecules, e.g. PEG oligomers
the targeting agent is selected from the group of small molecules, preferably folic acid (FA), octreotide (OCT) peptides, preferably LHRH, RGD, a monoclonal antibody, preferably Transtuzumab;
the complexing agent is selected from the group of diethylenetriaminepentaacetic acid (DTPA), 1,4,7,10-tetracyclododecane-N,—N′,N″,N′″-tetraacetic acid (DOTA), ethylene-diaminetetraacetic acid (EDTA), 1,4,7,10-tetraazacyclododecane-N,N′,N″-triacetic acid (DO3A), 1,2-diaminocyclohexane-N,N,N′,N′-tetraacetic acid (CHTA), ethylene glycol-bis(beta-aminoethylether)N,N,N′,N′,-tetraacetic acid (EGTA), 1,4,8,11-tetraazacyclotradecane-N,N′,N″,N′″-tetraacetic acid (TETA), and 1,4,7-triazacyclononane-N,N′,N″-triacetic acid (NOTA);
the metal ion is selected from the group of calcium, magnesium, copper, gallium, gadolinium and, manganese ion;
and formulating agent is selected from the group of glucose, physiological salt solution, PBS, or any combination thereof.

3. The composition according to claim 1, which is characterized by any one or more of the following features:
(i) the average size of the nanoparticles is in the range between 30 to 500 nm, preferably 60 to 200 nm, more preferably about 80 to 120 nm;
(ii) the proportion of the polycation to the polyanion is about 1:20 to 20:1 based on the weight of the agents;
(iii) the polyanion has a pH of 7.5 to 10; a molecular weight of 10 000 Da to 1.5 MDa and a concentration of 0.01 to 2 mg/ml;
(iv) the polycation has a pH of 3.5 to 6; a molecular weight of 60 to 320 kDa and a concentration of 0.01 to 2 mg/ml.

4. A process for the preparation of the composition according to claim 1, characterized in that it comprises the steps of
(i) a targeting agent is bound covalently to the polycation and/or the polyanion;
(ii) the active agent is bound covalently or by an ionic bond to the polycation and/or the polyanion;
(iii) the polycation and the polyanion are contacted with each other, preferably in a ratio of 1:20 to 20:1 based on the weight of the agents, thus are reacted with each other to self-assemble;
(iv) optionally the other components are added to the reaction mixture.

5. The process according to claim 4, wherein the polyanion used has a pH of 7.5 to 10; a molecular weight of 10 000 Da to 1.5 MDa and a concentration of 0.01 to 2 mg/ml; and the polycation used has a pH of 3.5 to 6; a molecular weight of 60 to 320 kDa and a concentration of 0.01 to 2 mg/ml.

6. A stable self-assembled composition comprising
(i) a carrier and targeting system comprising an optionally modified polyanion, and optionally a polycation, which may also be modified; at least one targeting agent which is linked to either the polycation/modified polycation or the polyanion/modified polyanion, or both;
(ii) paclitaxel as active compound and optionally
(iii) at least one complexing agent, metal ion and stabilizer/formulating agent or a PEGylating agent, which is obtainable by the process according to claim 4.

7. A pharmaceutical composition comprising the composition according to claim 1 together with pharmaceutically acceptable auxiliary materials, preferably selected from group of glucose, physiological salt solution, and PBS, or any combination thereof.

8. (canceled)

9. (canceled)

10. A method for the treatment of a subject in need for the treatment of tumours, especially human cervical adenocarcinoma, human ovary carcinoma, human breast carcinoma, human lung adenocarcinoma, human cervical carcinoma, human skin melanoma, human colon adenocarcinoma and human prostate carcinoma by administering to the subject an effective amount of the composition according to claim 1.
